(19) United States
(12) Patent Application Publication
(10) Pub. No.: US 2021/0310047 A1
(43) Pub. Date: Oct. 7, 2021

(54) Reaction Mixture, Method and Kit for Performing a Quantitative Real-Time PCR

(71) Applicant: Robert Bosch GmbH, Stuttgart (DE)

(72) Inventors: Sonja Kuellmer, Calw (DE); Tino Frank, Luzern (CH)

(21) Appl. No.: 17/264,631

(22) PCT Filed: Jun. 26, 2019
(86) PCT No.: PCT/EP2019/066957

§ 371 Date: Jan. 29, 2021

(30) Foreign Application Priority Data
Aug. 3, 2018 (DE) 10 2018 213 027.2

(51) Int. Cl. C12Q 1/686

(52) U.S. Cl. CPC C12Q 1/686

(57) ABSTRACT

A reaction mixture for providing a reaction batch for performing a quantitative real-time PCR contains at least one target DNA, which at least in parts corresponds to the DNA section being quantified, at least one reference DNA of defined sequence and in a defined amount, at least two different fluorescent probes of different sequence which generate a signal at different wavelengths, primers, deoxynucleotides and a DNA polymerase. The target DNA and the reference DNA have the same primer binding sites and different probe binding sites. At least one of the fluorescent probes is intended for binding to a section of the target DNA outside the primer binding sites in the amplicon, and at least one of the fluorescent probes is intended for binding to a section of the reference DNA outside the primer binding sites in the amplicon.

The present invention relates to a reaction mixture for providing a reaction preparation for performing a quantitative real-time PCR and also to a method for performing a quantitative real-time PCR and to a kit.

### PRIOR ART

The polymerase chain reaction (PCR) is a highly sensitive bioanalysis method. With the aid of the enzyme DNA polymerase, DNA is amplified on the basis of a DNA sequence as a model. The products formed in each cycle serve as a model for the respectively next cycle. The DNA to be multiplied is referred to as a template (template DNA). Furthermore, so-called primers, which each define the starting point of DNA synthesis on the individual strands of the DNA, are required. DNA synthesis is catalyzed by the temperature-stable DNA polymerase with use of deoxynucleotides. For each PCR cycle, the double-stranded DNA is first denatured (melting) before primer hybridization can take place, i.e., the binding of the primers to the complementary sequence segment of the single-stranded DNA (primer annealing). This is followed by attachment of the DNA polymerase, and complementary extension of the primers occurs in the so-called elongation step (extending). These individual steps are controlled by temperature cycles.

One embodiment of PCR is real-time PCR (qPCR), in which the reaction course can be followed by means of fluorescent probes. Real-time PCR allows a quantification of the starting amount of the template DNA. Generally, this requires reference measurements, which are concomitantly conducted in parallel reaction preparations. In addition to quantitative reference measurements, it is standard to concomitantly conduct qualitative controls as well in order to be able to rule out false-positive or false-negative results.

### DISCLOSURE OF THE INVENTION

### Advantages of the Invention

The invention provides a reaction mixture which is intended for providing a reaction preparation for performing a quantitative real-time PCR. With said reaction mixture, it is possible to carry out the quantification of a DNA sequence, for example a DNA sequence of a gene segment, the reaction preparation in question or the reaction mixture already having parallel standard reactions integrated therein, meaning that it is not necessary to concomitantly conduct parallel standard and control reactions. The particular advantage here is that the quantification and a quality control can be measured in the same reaction preparation (PCR preparation). In this connection, a reaction preparation is to be understood to mean that the reaction proceeds in one reaction vessel. It is thus not necessary that reference measurements be concomitantly conducted in parallel, thereby allowing considerable savings in the expenditure for PCR preparations. For example, this can provide considerable advantages especially in the case of point-of-care (PoC) applications, since tests are generally directly carried out for a patient in such applications. For this purpose, it is generally necessary in conventional methods to prepare relevant reference measurements for each patient and to concomitantly conduct them in parallel. This is not applicable when using the reaction mixture of the present invention. The reaction mixture of the present invention and the method performable therewith are therefore advantageously usable particularly in medical diagnostics.

The reaction mixture of the present invention comprises at least one target DNA which corresponds at least in parts to the DNA sequence to be quantified. Hereinafter, said target DNA is also referred to as a quanticon. Furthermore, the reaction mixture contains at least one reference DNA which has a defined, artificial sequence and which is present in a defined amount in the reaction mixture. Said reference DNA is hereinafter also referred to as an articon. Furthermore, at least two different fluorescent probes of different sequence that generate a signal at different wavelengths are provided. Furthermore, primers, deoxynucleotides and a heat-stable DNA polymerase are present. Depending on the application, the primers are one or more primer pairs. The target DNA and the reference DNA have the same primer binding sites (primer hybridization sites). Furthermore, different probe binding sites are provided on the target DNA and the reference DNA, said probe binding sites lying outside the primer binding sites in the respective amplicon. Here, the term amplicon generally refers to the DNA which is to be multiplied. At least one of the fluorescent probes is intended for hybridization or binding with a segment of the target DNA outside the primer binding sites in the amplicon. At least one of the fluorescent probes is intended for hybridization or binding with a segment of the reference DNA outside the primer binding sites in the amplicon. One of the fluorescent probes thus binds to the target DNA and the other fluorescent probe binds to the reference DNA. The fluorescent probes are preferably single-stranded DNA sequence segments which are each coupled to at least one reporter dye molecule and to at least one quencher molecule. The functional principle of such fluorescent probes known per se is based on the fact that the fluorescent signal is extinguished in an intact fluorescent probe or in an intact DNA molecule of the fluorescent probe due to the spatial proximity of the reporter dye molecule and the quencher molecule. During the PCR reaction, the fluorescent probes attach to the respectively complementary segments of the template DNA (outside the primer binding sites). During amplification, the DNA polymerase migrates along the template DNA on the strand to be copied and, in doing so, inevitably encounters the attached fluorescent probe. Owing to a 5′-3′ exonuclease activity of the DNA polymerase, the fluorescent probes are cut, with the result that the spatial proximity of the reporter dye molecules and the quencher dye molecules is terminated, thereby yielding a fluorescent signal. This measurable fluorescent signal can therefore indicate that amplification has taken place. By using two different fluorescent probes, one of which interacts with the target DNA or the other of which interacts with the reference DNA, it is therefore possible to follow, in one reaction preparation, both the amplification based on the target DNA and the amplification based on the reference DNA owing to the different fluorescent signals. Expediently, the fluorophores of the probes are chosen such that the colors or fluorescent signals are distinguishable from one another by means of a detector device and a suitable filter set.

The defined, artificial sequence of the reference DNA is expediently nonidentical (orthogonal), i.e., thus nonhomologous, in relation to the sequence of the target DNA, though the GC content, i.e., the overall proportion of guanine (G) and cytosine (C) in the sequence, independent of the positions thereof in the sequence itself, is preferably as identical as possible to the GC content of the target DNA. Here, “as identical as possible” is to be understood to mean that there can be a deviation of the percentage GC content of the target DNA and the reference DNA of, for example, up to 15%, preferably of up to 10%. Furthermore, it is preferred that the base pair length of the target DNA sequence and the reference DNA sequence is as same as possible, with deviations of, for example, up to 15%, preferably of up to 10%, being acceptable.

The concept for the performance of a quantitative real-time PCR, said concept underlying the described reaction mixture, is that an artificial reference DNA is added in a defined composition and amount to the reaction preparation. The reference DNA allows an internal calibration and can furthermore fulfill the functions of positive and negative controls. What is performed in principle is a multi-template PCR, yielding multiple different, specific amplicons in parallel in the amplification reaction. At least two templates, i.e., the target DNA and the reference DNA, are provided. The amplification of the different templates is, in principle, carried out using just one primer pair which hybridizes with the target DNA and the reference DNA. By using different probes, one probe being specific for the target DNA and one probe being specific for the reference DNA (or optionally more probes), it is possible to detect different fluorescent signals or fluorescent colors, and the assay result can be obtained from the ratios of the different fluorescent signals to one another.

Because of the integrated references and controls, a PCR process which is performed using the described reaction mixture is particularly suitable for automation and miniaturization, especially in the context of a microfluidic application. Here, it may be particularly advantageous if the different components of the reaction mixture are provided in lyophilized form. Thus, especially the target DNA and/or the reference DNA and/or the primers and/or the deoxynucleotides and/or the DNA polymerase can be provided and initially charged in lyophilized form. This can, for example, be realized in the form of one or more so-called lyobeads. A lyobead is generally to be understood to mean a lyophilisate which has been pressed into a spherical shape after production, after which the substances are generally present as powder. For example, the components necessary for the PCR preparation can be provided in lyophilized form, especially the DNA polymerase, the deoxynucleotides, the target DNA and the reference DNA and the reaction buffer components and optionally also the primers and/or the probes. In this way, the PCR process can be directly started in a very user-friendly manner by addition of the sample to be quantified and optionally of further necessary components. Provision in lyophilized form is very advantageous especially for automated applications.

The provision of the reaction mixture or at least parts of the reaction mixture as lyobead has furthermore the advantage that the integration of standards and/or controls in one reaction preparation can considerably reduce the effort of production and also the effort of development for the lyobeads. Owing to the reduction in the number of necessary reaction preparations, integration into a microfluidic system is also particularly advantageous, since fewer reaction chambers are necessary than in the case of conventional PCR processes and the microfluidic platform need not be expanded by further chambers. Furthermore, the run time of the real-time PCR can be shortened, since the concept underlying the invention makes it possible for the reaction conditions to be brought into a particularly efficient or ideal reaction range by predefined amounts of the templates, with the result that fluorescent signals can always be expected.

A further particular advantage of the presently described PCR process is that, owing to the integration of standards and/or controls in one reaction preparation, the conditions for the standards, controls and the actual sample containing the DNA to be quantified are identical. If, for example, an air bubble is present in the reaction preparation, which may be the case in rare cases, for example in microfluidic systems, the associated effects on reaction efficiency are identical for all templates, i.e., for example for the quality control, the calibration and for the actual sample reaction, meaning that the entire experiment is comparable and evaluable in any case.

Conventionally, a standard curve is often created as part of a quantitative real-time PCR, this often requiring at least three different concentrations for the standard curve, which concentrations are in the range of the sample concentration to be expected. For statistical reasons, more concentrations are often chosen for the standard reactions and said standard reactions are moreover processed in replicate. In the case of the presently described concept for real-time PCR, the calibration is carried out by means of a multi-signal concept of the different fluorescent probes and the ratio thereof to one another, and this is why only one reaction is required and a quantification is possible nevertheless. This has considerable advantages with respect to the amount of work and processing required therefor and also with respect to an advantageous minimization of costly chemicals and to the sample requirement or the necessary low amount of sample.

In a preferred embodiment of the reaction mixture, the amount of the reference DNA can be present in a concentration which corresponds to a detection limit for the DNA segment to be quantified. Furthermore, depending on the application, the target DNA and the reference DNA can be present in a ratio of 1:1 and additionally in defined amounts.

The invention furthermore encompasses a method for performing a quantitative real-time PCR, said method using at least one reaction mixture as described above. What is additionally added to said reaction mixture is generally the actual sample containing the nucleic acid material which (possibly) comprises the DNA segment to be quantified. With this completed reaction preparation, the PCR process is, in a way, performed as a duplex reaction, the PCR cycles being performed in a manner known per se by varying the temperature in a thermocycling process known per se. This involves amplification of, firstly, the target DNA and the DNA segment to be quantified, if present in the sample, and, secondly, the reference DNA. By capturing and evaluating the fluorescent signals of the different fluorescent probes, which are respectively specific for the target DNA (and at the same time for the DNA segment to be quantified) and the reference DNA, it is possible to capture and distinguishably track the amplification of the target DNA and, at the same time, of the actual sample containing the DNA segment to be quantified and the amplification of the reference DNA. From the ratio of these signals to one another, it is possible to ascertain an assay result and especially a quantitative assay result.

In a particularly preferred embodiment of the method, the method is performed in a PCR array comprising a plurality of array vessels. This can be done particularly advantageously in microfluidic applications, which are especially also amenable to automation. Here, each array vessel of the PCR array can be loaded with different reaction mixtures, and a maximum degree of multiplexing is therefore possible. The loading can, for example, be achieved by spotting each array vessel with a different reaction mixture. What is possible as a result is that, especially in a microfluidic PCR array, the sample solution containing the DNA segment to be quantified or the nucleic acid material to be tested can, for example, be added across the array as a whole. In this way, the sample solution reaches each individual vessel and forms with the respectively different reaction mixture a respective reaction preparation. The particular advantage here is that the individual reaction chambers need not be individually filled and actuated. In conventional methods, there is the problem that, in the case of a PCR array, the array vessels which are intended for the standard reactions must not be loaded with sample material. In this respect, it is generally necessary in conventional PCR arrays that the individual reaction chambers be individually filled and actuated, with the reaction vessels for the standard reactions being filled differently than the reaction chambers which are intended for the PCR processes with the actual sample. By contrast, the PCR array with which a PCR process according to the presently described concept is performed allows, firstly, a substantially larger number of PCR preparations containing the sample to be measured in one array, since separate reaction preparations do not need to be provided for the standard reactions. Secondly, the concept of the present application furthermore allows, as described above, filling of the entire array as a whole with the sample solution.

A further particular advantage of the presently described concept for a real-time PCR is that the reaction system need not be calibrated for each light source, since the assay results are taken from the ratio of the signals, which is based on the conserved ratios in the individual amplification runs. Light sources and optical detectors often differ in different instrument types. Therefore, calibration measurements are conventionally necessary for every instrument type. Even in one instrument in which two identical LED light sources are installed, it is conventionally necessary to calibrate both light sources so that they provide the same absolute numbers necessary for the evaluation via standard curves. In the case of the concept for a real-time PCR according to the present invention, these complicated calibration measurements are not applicable, since the procedure involves relative ratios within one preparation.

In the case of medical applications and especially in medical diagnostics, the sample amounts which are obtained from the patient are often small. Furthermore, analytical systems which are used in point-of-care applications are intended for a small space requirement and should have a highest possible degree of automation in order to reduce the complexity of operation. In this respect, especially microfluidic realizations of the presently described PCR preparation are particularly suitable for these applications, with automation, miniaturization and parallelization being possible, this reducing the complexity of use and minimizing the potential for error with operating errors. Furthermore, it is possible to transfer small sample amounts in small volumes, meaning that the reaction concentration becomes greater. In the case of conventional methods, parallelization is associated with handling challenges, since the distribution of reaction mixtures and the prestorage and processing of the necessary chemicals is generally difficult owing to miniaturization. The real-time PCR according to the presently described concept minimizes the complexity in the handling of the PCR process, since, firstly, the number of reaction preparations is reduced to essentially one preparation. Here, the necessary reagents can be readily prestored, for example in a lab-on-a-chip system. Through the option of lyophilization, the reagents can, for example, be provided even at room temperature and on the smallest space, for example in the form of lyobeads.

In a particularly preferred embodiment of the method, the method can be used for a nested PCR process. A nested PCR process comprises, in a manner known per se, a preamplification and at least one downstream detection reaction. Here, the presently described concept can, with use of a target DNA and a reference DNA in one preparation, be used for an estimation of the amount of PCR product of the preamplification. Here, the target DNA and the reference DNA can be designed such that a first primer pair is used for the preamplification and at least one second primer pair is used for the detection reaction(s). The target DNA and the reference DNA each have complementary sequence segments in relation to the primer sequences (primer binding sites), with the complementary sequence segments for the second primer pair (primer binding sites for the primer pair of the detection reaction(s)) lying within the complementary sequence segments for the first primer pair (primer binding sites for the primer pair of the preamplification). This means that the primer binding sites for the individual primer pairs are, in a way, nested in one another on the target DNA and the reference DNA.

The nested PCR process can be used especially for a point mutation detection. Here, after the preamplification, the amount of PCR product of which is determined according to the presently described concept, it is possible to perform the detection reaction using a mutation-sensitive primer and/or a mutation-sensitive fluorescent probe.

The nested PCR process can be a multiplex process in which at least two particular gene segments in a genome are to be detected. Here, what can be performed for a quantification of the preamplification is a control reaction in which a control exon from the genome is amplified in parallel. In this case, the target DNA and the reference DNA are matched with the control exon, and what are deduced from the quantification of the amplification of the control exon according to the presently described concept are the amounts in the case of the amplification of the gene segments to be detected during the preamplification.

Altogether, the presently described concept for a PCR process can integrate not only quantitative standard curves and quality reactions, but also reference and threshold measurements, which are required, for example, in point mutation assays in oncology. Although detection requires two color channels per DNA segment to be tested, the concept allows multiplexing, and it is possible to address multiple different DNA segments (targets) in one preparation. Especially in the context of a nested PCR comprising preamplification and a downstream qualitative measurement, for example an analysis of a point mutation, it is possible to apply the method such that the starting amount for the second reaction is estimated by a quantification of the preamplification. At the same time, the two PCR processes, i.e., the preamplification and the downstream detection reaction, can be linked to one another in a fully automated manner in a microfluidic system without the DNA concentration having to be measured separately in an intermediate step or without PCR products which arise in the preamplification having to be purified. The nested PCR process can, for example, be embodied such that the estimation of the amount of PCR product from the preamplification is followed by setting of an optimal DNA concentration for the subsequent detection reaction(s) by dilution. This can, for example, be performed in situ, in an automated manner as well.

The invention lastly encompasses a kit for performing a quantitative real-time PCR. The kit comprises at least one target DNA which corresponds at least in parts to the DNA sequence to be quantified. Furthermore, the kit comprises at least one reference DNA having a defined, artificial sequence and in a defined amount. Furthermore, there are at least two different fluorescent probes of different sequence that generate a signal at different wavelengths. Optionally, primers and/or deoxynucleotides and/or a DNA polymerase and/or buffer components can be provided. The target DNA and the reference DNA have the same primer binding sites, but different probe binding sites, said probe binding sites lying outside the primer binding sites in the respective amplicon. At least one of the fluorescent probes is intended for hybridization (binding) with a segment of the target DNA outside the primer binding sites in the amplicon and at least one of the fluorescent probes is intended for hybridization (binding) with a segment of the reference DNA outside the primer binding sites in the amplicon. The components of the kit can be provided especially in lyophilized form, for example in the form of lyobeads. With regard to further features of said kit, reference is made to the above description.

Further features and advantages of the invention are apparent from the following description of exemplary embodiments. Here, the individual features can each be realized separately or in combination with one another.

In the figures:

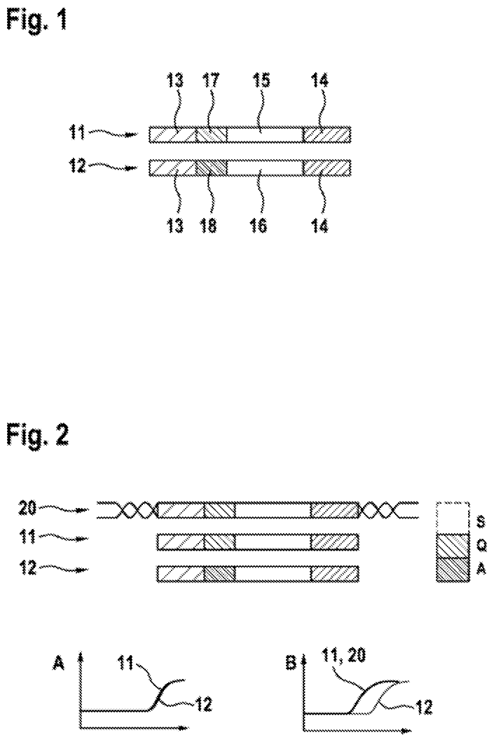

FIG. 1 shows a schematic representation of the design of the target DNA and the reference DNA to illustrate the basic principle of the concept for performing a quantitative real-time PCR;

FIG. 2 shows a schematic representation of the template DNAs used for the quantitative real-time PCR and a schematic representation of possible experimental results in the quantification of a particular DNA segment in a sample;

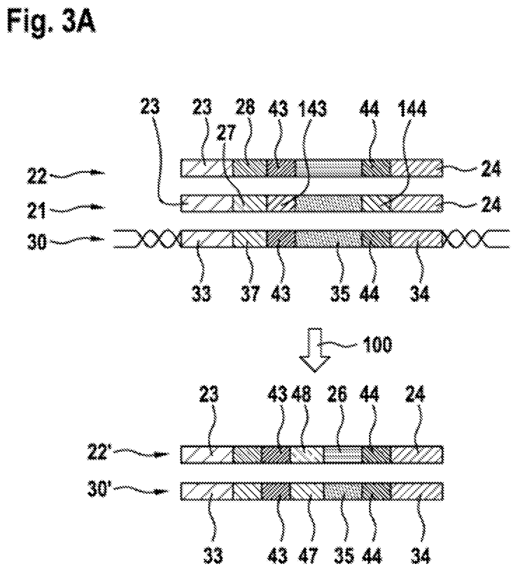

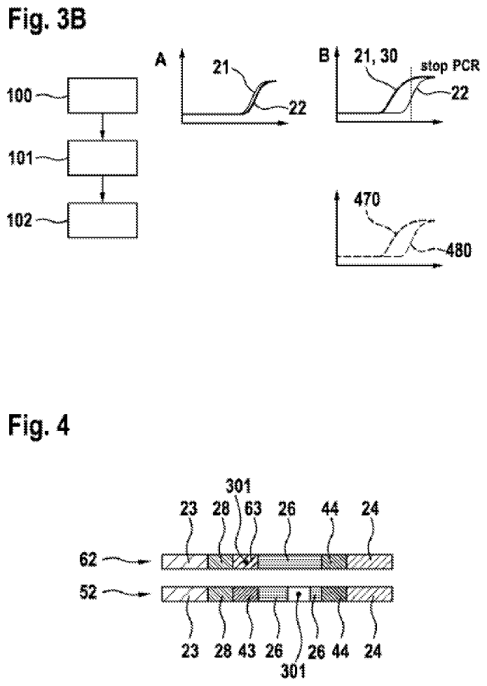

FIG. 3 shows a schematic representation of the DNA templates used for a quantitative real-time PCR (FIG. 3A) and a schematic representation of possible experimental results (FIG. 3B) in the application of the concept in the context of a quantitative nested PCR;

FIG. 4 shows a schematic representation of possible designs for a reference DNA in the context of a point mutation assay;

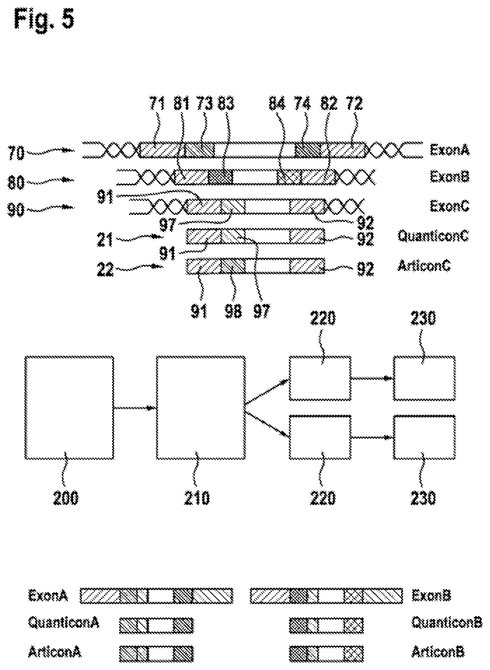

FIG. 5 shows a schematic representation of the template DNAs used to elucidate a multiplex embodiment of a nested PCR and

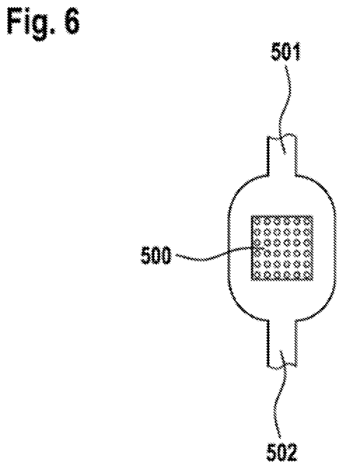

FIG. 6 shows a schematic representation of the implementation of the quantitative real-time PCR in a microfluidic PCR array.

### DESCRIPTION OF EXEMPLARY EMBODIMENTS

FIG. 1 elucidates the basic principle of the design of the template DNAs used, i.e., the target DNA 11 and the reference DNA 12. What can be used as the basis for the PCR reaction preparation is a classic TaqMan® system, with use of two different fluorescent probes, as elucidated at the start. Here, the target DNA corresponds to the DNA sequence to be actually analyzed or quantified, for example the DNA sequence of a gene segment. The target DNA 11 is supplemented with an artificial reference DNA which has a defined sequence and which is used in a defined amount. The target DNA 11 and the reference DNA 12 have the same primer binding sites, i.e., respectively a binding site 13 for the forward primer and respectively a binding site 14 for the reverse primer. In the rest of the base pair sequence 15, 16, these template DNAs 11, 12 differ. In particular, they have different binding sites 17, 18 for the probes used. The two template DNAs 11 and 12 are also referred to as quanticon 11 for the amplicon to be quantified and as articon 12 for the artificial amplicon. The following table summarizes the design of the quanticon (target DNA) 11 and the articon (reference DNA) 12:

| | Quanticon | Articon |
|---|---|---|
| Forward primer | Target-specific | Target-specific |
| Probe | Target sequence, | Orthogonal sequence, |
| | fluorophore of | fluorophore of |
| | color A | color B |
| Sequence | Target sequence | Orthogonal sequence |
| Reverse primer | Target-specific | Target-specific |

The fluorophores of the fluorescent probes are chosen such that the two colors are distinguishable from one another by means of a detector (filter set). The orthogonal sequence 16 of the reference DNA 12 is expediently nonidentical in relation to the target sequence 15 of the target DNA 11. The GC content should be as identical as possible to the GC content of the target sequence 15 of the target DNA. The base pair length of quanticon, i.e., target DNA 11, and articon, i.e., reference DNA 12, should be of the same length as well. As a result, the melting temperatures of the two amplicons 11, 12 are very similar, and so the same amounts of amplified material arise in principle in an efficient PCR. Using these template DNAs, a quantitative real-time PCR is carried out, with amplification of quanticon 11 and articon 12 in the same reaction vessel as an effectively duplex reaction. Meanwhile, the two probes are recorded, for example after each PCR cycle or continuously. Here, the articon 12 can be initially charged in a predefined amount in the region of or above the detection limit and must be detected as signals of the probe B in the event of a successful PCR. The articon serves in this case as a reaction control. The amplification of the quanticon 11 and of the gene segment (target) to be quantified possibly additionally present in the reaction preparation is detectable as a signal of probe A. The signals of probe A and B are, then, in defined ratios. If the same starting amount of articon 12 and quanticon 11 is present, the two amplification curves are congruent. If more quanticon 11 is present, it is detected earlier and the curve of the articon 12 follows depending on the concentration thereof. This can be calculated by means of the reaction efficiency and from the firmly defined amount of the articon 12. The initially charged amount of articon 12 is, then, an absolute reference point, the amount of which is known. The efficiency of the reaction can be ascertained by means of the curve shape of the exponential phases. As a result, the unknown starting amount of the quanticon 11 can be calculated using the absolute reference point.

FIG. 2 shows the implementation of the reaction system for the case of the sample material (sample) being present as a genome. For example, this may be used if particular gene segments from a lysate are to be detected. Comparable with the principle from FIG. 1, what is used here is a quanticon 11 (Q) and an articon 12 (A). Additionally present in the reaction preparation is the gene segment 20 (S) to be amplified, as cell lysate containing genetic material which is formed by the genome of the cell(s). In this case, the quanticon 11 and the articon 12 are initially charged in a predefined amount in a ratio of 1:1. The amount chosen can, for example, lie in the proximity of above the detection limit. Another possibility is to adjust the amount to an ideally functional range for the PCR reaction, so that the PCR proceeds particularly efficiently. In general, every quantitative real-time PCR (qPCR) has limits within which the reaction proceeds efficiently. In this connection, the detected C_{T} values, which describe the start of the exponential growth of a curve, are in a linear ratio to the logarithmized starting amount used. If the amount used of the quanticon 11 and the articon 12 is chosen in this range, a signal should be detected with each successful PCR process. The signal of the articon 12 is the signal which must be measured last in chronological order. If it is missing, the reaction control is negative. If the signal of the quanticon 11 is detected simultaneously with the signal for the articon 12, this means that only quanticon 11 and articon 12 was present in the reaction mixture and no sample 20. This case is represented in graph A of FIG. 2 and serves as a detection control for the function in principle of the PCR preparation. Here, the lines 11 and 12 represent the respective fluorescent signals of the quanticon 11 (fluorophore A) and the articon 12 (fluorophore B). If the same DNA segment as in the quanticon 11 was present in the genome or in the sample 20, said segment from the sample 20 is concomitantly highly amplified. The result of this is that the signal of the quanticon 11 (graph B in FIG. 2) is detected earlier, the detected signal being composed of the amplification of the quanticon 11 and the sample 20. As already elucidated in the principle from FIG. 1, it is possible, then, to calculate the starting amount of the DNA segment to be tested from the sample 20 (sample). The predefined amounts of articon 12 and quanticon 11 provide, then, not only the absolute reference point to calculate the quantification, but also ensure signals and serve as control.

In a further embodiment of the PCR process, the process can be performed dynamically, by the signal of the quanticon 11 representing a termination criterion for the reaction, so that the PCR process can be ended after the appearance of the signal of the quanticon 11. Since the amount of the quanticon 11 can be transferred into an efficient range for the PCR process, what is possible is a detection approximately in the temporal middle of the planned process duration, i.e., in middle cycle numbers. In the event of a positive sample, i.e., the sought DNA segment in the sample 20 is present, the signal of the quanticon 11 (together with the signal of the sample 20) lies before the signal of the articon 12, and so the process time for measurement can be shortened. In this case, only a qualitative statement is possible after the termination of the reaction.

FIG. 3A and FIG. 3B illustrate the described concept in the context of a qualitative nested PCR. Such a PCR method can, for example, be used for detection of mutations. To this end, what is highly copied from the genomic DNA 30 of cells is the target region in which the mutation lies. Thereafter, the ratio of wild type to mutation is measured. For this purpose, a preamplification is upstream of the actual detection reaction in order to ensure that sufficient material is present for a detection. This is especially of great significance if little cell material is present, such as, for example, in the case of a liquid biopsy containing circulating tumor cells. As illustrated in FIG. 3A, the presently described concept is implemented in such a system such that the locus of the mutation 35 (target DNA) is initially highly copied in a sufficiently large segment using a defined primer pair. For this first primer pair, what are present on the genomic DNA of the sample 30 are corresponding binding sites 33, 34 for a forward primer and a reverse primer. For the control, monitoring and quantification of the process, what is chosen is a probe binding site 37 for a first fluorescent probe A in immediate proximity of the primer binding site 33. Designed congruently with this amplicon in the sample 30 is a quanticon 21, i.e., a target DNA 21 which has corresponding primer binding sites 23 and 24 and a corresponding probe binding site 27 for the fluorescent probe A. Additionally designed is an articon 22 (reference DNA) having the same primer binding sites 23, and a differing probe binding site 28 for a fluorescent probe B. These components 30, 21, 22 provide the basis of the preamplification, which is quantifiable according to the principle elucidated by means of FIG. 2. Furthermore, for the subsequent detection reaction 102, what are provided are further primer binding sites 43, 44 for a further primer pair comprising a second forward primer and a second reverse primer, the binding site 43 for the second forward primer being joined to the binding site 37 for the fluorescent probe A in the case of the genomic gene segment 30. The binding site 44 for the second reverse primer is situated downstream of the actual target DNA 35 which represents the gene segment to be detected. Situated on the reference DNA 22 (articon for the preamplification) are corresponding primer binding sites 43, 44. Provided on the target DNA 21 (quanticon for the preamplification) are differing, i.e., orthogonal, sequences at the positions 143, 144 which correspond to the primer binding sites 43, 44 of the articon sequence 22. The sequence between the sequences 143, 144 on the quanticon sequence 21 corresponds to the target DNA sequence 35 of the DNA segment to be quantified of the sample 30.

After the preamplification 100, which is carried out after addition of the first primer pair, what is present as PCR product is the amplified gene segment 30′ to be quantified (amplified sample). Additionally present is the amplified articon 22′. The likewise amplified quanticon 21 substantially corresponds, from the sequence, to the amplified sample 30′. Within the primer binding sites 43, 44 for the second primer pair of the subsequent detection reaction 102, what is situated after the primer binding site 43 for the forward primer is a binding site for a further fluorescent probe A′ which is used in the subsequent detection reaction 102. Correspondingly, the articon 22 or the amplified articon 22′ has, after the primer binding site 43, a different probe binding site 48 for a further fluorescent probe B′, likewise for the subsequent detection reaction 102. On the articon 22 or the amplified articon 22′, what follows is an orthogonal sequence 26 which is orthogonal in relation to the target sequence 35 of the sample 30 to be tested. What follows is the binding site 44 for the reverse primer of the subsequent detection reaction 102 and the binding site 24 for the reverse primer from the preamplification. The GC content and the length of the base pair sequences among articon 22 and the corresponding segment in the sample 30 and the quanticon should approximately correspond, as already explained above.

The quantification of the preamplification 100 is, in principle, carried out as already elucidated by means of FIG. 2 and is illustrated in the top part of FIG. 3B. The signals of the probes A and B are both depicted here. Graph A shows the case of overlapping of the signal of the quanticon 21 (probe A) and the signal of the articon 22 (probe B). In this case, there is no DNA segment to be detected in the sample 30. Graph B shows the case of the signal of the amplified quanticon 21 together with the amplified gene segment from the sample 30 appearing chronologically before the signal of the amplified articon 22. In this case, the sought gene segment in the sample 30 is present. If no sample can be detected (graph A), the entire run can be stopped and what can be output is that no detection occurred (negative assay result). If, as per graph B, sample is detected, the PCR process can be continued until the articon 22 is detected. Then, the process can optionally be terminated. Alternatively, a predefined number of PCR cycles can also be executed. From the amplification curve of the sample 30 together with the quanticon 21, it is possible to calculate efficiency. By means of the predefined articon 22, it is possible to calculate the end concentration and the start concentration of all amplified materials. On this basis, the preparation can be diluted and be prepared with a new master mix (step 101) in such a way that the preparation corresponds to the ideal starting concentrations for the subsequent detection assay(s) (step 102). The dilution can be done by hand, for example when the reactions take place in a bulk system, for example a classic qPCR cycler. Preferably, the process is carried out in a fully automated liquid handler, microfluidic systems being particularly suitable. Here, liquids can be diluted and distributed with the aid of microfluidic pumping and aliquoting systems.

For the actual detection reaction 102, for example for a point mutation detection, the reaction preparation is amplified using the primers required for this purpose (second primer pair) and the signal course of the probe A′ (curve 470) and the probe B′ (curve 480) is observed and evaluated. According to the reaction concept, the articon 22′ is outnumbered, i.e., less starting material of the articon 22′ is present than starting material of the sample 30′. This is because more sample amplicon, consisting of the amplified sample 30′ and the amplified quanticon 21, arises in the preamplification 100. Therefore, a dynamic termination of the PCR after immediate detection is highly advantageous, since the articon 22′ and the sample 30′ in the exponential phase make the estimation of the amplicon amounts more accurate than in the case of a detection in the saturation phase. Since, then, the articon 22′ is again present in defined amounts and the sample 30′ acts as a new quanticon, the second qPCR, i.e., the detection reaction 102, can also be completely quantified. The number of copies from the start up to the end of the process is thus known. The amplified quanticon 21 of the first reaction (preamplification 100) does not come into consideration in the second reaction (detection reaction 102), since the corresponding positions 143, 144 in relation to the primer binding sites 43, 44 on the target DNA sequence 21 of the preamplification were chosen orthogonally, i.e., differingly.

For the detection reaction 102, it is possible to add to the master mix thereof additionally a further articon. Here, instead of the articon from the first preamplification, what is added is a new articon for the second detection reaction. This is useful, since the first reaction mixture is generally diluted and therefore the articon (but not the increased actual sample) is detected. Therefore, after the dilution, a defined amount of articon is added again for a more accurate determination. This further articon can, for example, be prestored in a (second) lyobead required for the detection reaction. This is especially advantageous for determining the ratio of wild type and mutation type in a point mutation detection. A further quanticon which has the same primer binding sequences is also used. The amplified material of the first reaction 100 must then be diluted such that said reaction corresponds to the concentration of the initially charged, second quanticon.

FIG. 4 shows embodiments for a possible design of the articons (reference DNA) 52, 62 for a point mutation detection. Said articons 52, 62 are intended for the application of a nested PCR in the context of a point mutation assay. Generally, two general PCR detection strategies are used in point mutation detections. What are chosen here are either mutation-sensitive primers (a) or mutation-sensitive probes or blockers (b). In method (a), the primer is designed such that it can only bind when the mutation is present. Examples thereof are so-called ARMS (amplification refractory mutation system) systems. In method (b), what is used is a mutation-sensitive probe or blocker which only binds when the mutation is present (e.g., PNA-CLAMP systems—peptide nucleic acid (PNA)-mediated PCR clamping; H. Ørum et al., Nucleic Acids Res. 21: 5332-5336, 1993). However, since these bindings are not 100% efficient, a reference signal in relation to the wild type is concomitantly measured. Therefore, for the implementation of the concept according to the invention for such a detection, it is useful to involve a second quanticon. For the implementation, the mutation 301 is incorporated into the orthogonal sequences for the articon 52, 62. The binding site of the mutation should therefore be included. In version 62 with a mutation-sensitive primer, the articon therefore comprises the following segments: binding site 23 for the first forward primer, binding site 28 for the probe B, binding site 63 for a mutation-specific primer which represents the forward primer of the second primer pair for the detection reaction 102, an orthogonal sequence 26, a binding site 44 for the reverse primer of the detection reaction 102 and a binding site 24 for the reverse primer of the preamplification. For a detection system with a mutation-sensitive blocker or a mutation-sensitive probe, the articon 52 is designed such that the binding site for this probe or the blocker comprises the mutation site 301 at exactly the same site as in the mutation type. Apart from that, the articon 52 corresponds to the articon 62 or the articon 22. Using such a construct, it is possible, then, to measure in the reaction a signal for a reaction with 100% wild type (second quanticon) and a signal for 100% mutation (articon 52 or 62) and to compare them with the sample. This is all possible within one reaction preparation, thereby allowing an extreme simplification of full automation, for example in a point-of-care application. It is particular advantageous here when the corresponding master mixes are initially charged as lyophilisates.

FIG. 5 shows a multiplex embodiment of a nested PCR, wherein two detection reactions can be performed in one preparation. In said embodiment, the starting point is a lysate composed of few cells, for example 10 to 1000 cells. What can be present in said lysate are, for example, cells from an enrichment, for example from an enrichment of circulating tumor cells or from an enrichment of immune cells, for example specific T cells, from a body fluid, such as blood, urine, spinal fluid or other. Said cells are lysed in a small volume and provide the sample for the performance of the method. In said cell lysate, two gene segments 70, 80 are to be detected, i.e., for example an exon A (gene segment 70) and an exon B (gene segment 80). The gene segments 70, 80 are initially amplified (preamplification), so that it is possible in the downstream step to detect, in one or more detection reactions, anomalies on said gene segments such as mutations or function-typical gene sequences, for example the genetic coding of an antigen epitope. In the first step of the preamplification, the two target exons, i.e., the gene segments 70 and 80, are first amplified from the genetic material of the lysed cells. In addition, a sample control is run. For the sample control, a control exon C is amplified as gene segment 90. Here, this can, for example, be an exon of the sought gene, on which the anomaly is not present. If it is amplified, the reaction is considered successful and to be evidence of the presence of the sample material, i.e., genetic material, in the sample. For the detection and the quantification of the amplification of the control exon 90, what are correspondingly used as already described are a target DNA (quanticon) 21 and a reference DNA (articon) 22 which are, from their structure and their components, matched with the control exon 90 according to the above-described principles. If the amplicons 70, 80 and 90 all have approximately the same length and the same GC content, then what should arise in the reaction preparation in a triplex reaction are about the same number of copies for each template. If there are deviations in the length and in the GC content, conserved ratios of the amplified amplicons ensue, it being possible for the ratios to be additionally influenced by the DNA structure and epigenetic modifications. This means that, even if the reaction is possibly less efficient in the case of the amplification of one of the exons, the ratios of the amplicon copies which arise are nevertheless constant. This allows measurement of just one amplicon, namely the control exon C, with respect to a quantification, and from this, it is possible to determine the amplicon number for all exons. A complicated three-probe design is thus not necessary; instead, it is generally sufficient to use just one two-probe system for the quantification of the control exon C (gene segment 90). Therefore, the preamplification is initially quantified by the control exon 90, and so the amplified materials can be estimated for the subsequent detection reaction as described above and optionally be distributed and diluted in situ for optimal reaction conditions in the detection reaction. After distribution and dilution, a new master mix is added which is intended for the specific assay of the detection reaction and which can likewise be quantified as per the remarks in relation to FIG. 2.

The design for the individual amplicons preferably looks as follows: Exon A (gene segment 70) has, on the periphery of the amplicon, the binding sites 71, 72 for the primers of the preamplification. Exon B (gene segment 80) and the control exon C (gene segment 90) have corresponding primer binding sites 81, 82 and 91, 92, respectively, but with different sequences. In the case of the exons A and B to be tested in the subsequent detection reaction, what follows in each case is the binding site for the primers of the second reaction (detection reaction) 73, 74 and 83, 84, respectively, on the gene segments 70 and 80, respectively. If a probe is intended for the detection reaction, the binding site thereof is included in this sequence. The control exon C (gene segment 90) has, after the primer binding site 91, the binding site 97 for a fluorescent probe A. Correspondingly as elucidated by means of FIG. 2, the quantification of the control exon C (gene segment 90) is achieved by supplementing a quanticon or target DNA 21 and an articon or reference DNA 22 that are provided with the same primer binding sites 91, 92 as the control exon C (gene segment 90). The target DNA 21 has furthermore the same binding site 97 for the probe A. The reference DNA 22 likewise has a probe binding site 98, but with a different sequence for binding a fluorescent probe B. From the signals to be generated with this reaction preparation, the resultant copies N_{C} and the starting quantity N_{O} e deduced. From the conserved ratios, the amounts of the exons A and B (gene segments 70 and 80) are calculated. Said ratios can be worked out as part of assay development. The ratios are specific for the assay in question. The ratios are intrinsically constant, but must be measured, i.e., parameterized, for each application. According to the method elucidated by means of FIG. 2, the master mixes of the subsequent two separate and parallelly processible specific detections for the exon A and for the exon B can each have a quanticon and an articon that have the same primer binding sites as the respective target exon A and B. For the detection of point mutations, it is possible, as elucidated by means of FIG. 4, for the quanticon to have the wild-type sequence and the articon to have the mutant sequence. The middle part of the representation in FIG. 5 schematically represents the entire reaction procedure. What takes place first is the preamplification 200, with the presence of exon A, exon B, control exon C and the quanticon and the articon as templates in the preparation. After the performance of the amplification reactions, what is obtained is the quantification result 210 (N₀ for control exon C, N_{C} for control exon C; calculable therefrom: N_{C}, N₀ for exon A, N_{C}, N₀ for exon B). To achieve optimal starting conditions for the subsequent detection reaction for the exon A and the exon B, the optimal concentrations of exon A (N_{S, 2} exon A) and exon B (N_{S, 2} exon B) are set in step 220 by distribution and optionally dilution of the preparations. Thereafter, by addition of the master mixes for the respective detection reactions, what is performed in step 230 is, for example, the respectively specific mutation detection, with not only the exon A and the exon B, but in each case a correspondingly designed quanticon A and articon A and quanticon B and articon B, respectively, being added for this purpose, as illustrated in the bottom part of FIG. 5.

FIG. 6 illustrates the implementation of the described PCR concept in a microfluidic qPCR array 500. The array 500 is, for example, integrated into a chip composed of structured silicon, said array 500 being situated in a microfluidic chamber which is provided with an inflow 501 and an outflow 502. In one possible embodiment, individual reaction vessels of the array 500 can be actuated, or admixed with liquids, in a global manner. For example, a preamplified sample can be flushed across the array 500, so that the individual reaction vessels of the array 500 are filled. By means of a seal, it is possible to prevent communication via diffusion between the individual reaction vessels in a second fluidic step. If, then, each reaction vessel of the array 500 is, for example, prespotted with a lyophilized master mix and/or with the primers and the probe sequences, what is possible by means of the method with an n×m array is a maximum n×m degree of multiplexing including a quantification and quality control. The reaction preparations according to the concept of the invention can preferably be developed on the basis of TaqMan® systems. The synthesis of the individual template DNAs, especially the quanticons and the articons, can be done using customary nucleic acid synthesis. Preferably, the master mixes including the template DNAs can be prestored as lyophilisates. Microoptofluidic systems in particular are suitable for an automation of the processes.

1. A reaction mixture for providing a reaction preparation for performing a quantitative real-time polymerase chain reaction (PCR) for quantifying at least one deoxyribonucleic acid (DNA) segment, the reaction mixture comprising:
at least one target DNA which corresponds at least in parts to the at least one DNA segment to be quantified;
at least one reference DNA having a defined sequence and in a defined amount;
at least two different fluorescent probes of different sequence that generate signals at different wavelengths; and
primers, deoxynucleotides, and DNA polymerase,
wherein the at least one target DNA and the at least one reference DNA have the same primer binding sites and different probe binding sites,
wherein at least one of the fluorescent probes is intended for binding with a segment of the at least one target DNA outside the primer binding sites, and
wherein at least one of the fluorescent probes is intended for binding with a segment of the at least one reference DNA outside the primer binding sites.

2. The reaction mixture as claimed in claim 1, wherein a GC content of the at least one target DNA and a GC content of the at least one reference DNA are identical with a deviation of up to 15%.

3. The reaction mixture as claimed in claim 1, wherein a base pair length of the at least one target DNA and of the at least one reference DNA are identical with a deviation of up to 15%.

4. The reaction mixture as claimed in claim 1, wherein the at least one target DNA, the at least one reference DNA, the primers, the deoxynucleotides, and/or the DNA polymerase are provided in lyophilized form.

5. The reaction mixture as claimed in claim 1, wherein the amount of the at least one reference DNA is present in a concentration which corresponds to a detection limit for the at least one DNA segment to be quantified.

6. The reaction mixture as claimed in claim 1, wherein:
the mixture is used to detect and optionally to quantify the at least one DNA segment from a genome, and
the reaction preparation contains the at least one target DNA and the at least one reference DNA in defined amounts in a ratio of 1:1.

7. A method for performing a quantitative real-time polymerase chain reaction (PCR), comprising:
performing a PCR process using at least one reaction mixture, wherein a sample containing at least one deoxyribonucleic acid (DNA) segment to be quantified is added to the at least one reaction mixture; and
capturing fluorescent signals of at least two fluorescent probes,
wherein the at least one reaction mixture comprises:
at least one target DNA which corresponds at least in parts to the at least one DNA segment to be quantified;
at least one reference DNA having a defined sequence and in a defined amount;
the at least two different fluorescent probes which are of different sequence and generate the fluorescent signals at different wavelengths; and
primers, deoxynucleotides, and DNA polymerase,
wherein the at least one target DNA and the at least one reference DNA have the same primer binding sites and different probe binding sites, and
wherein at least one of the fluorescent probes is intended for binding with a segment of the at least one target DNA outside the primer binding sites, and
wherein at least one of the fluorescent probes is intended for binding with a segment of the at least one reference DNA outside the primer binding sites.

8. The method as claimed in claim 7, further comprising:
ascertaining an assay result from a ratio of the fluorescent signals of the fluorescent probes.

9. The method as claimed in claim 7, further comprising:
performing the method in a PCR array comprising a plurality of array vessels.

10. The method as claimed in claim 7, further comprising:
using the method for a nested PCR process comprising a preamplification and at least one downstream detection reaction; and
estimating an amount of PCR product of the preamplification by the quantification.

11. The method as claimed in claim 10, wherein:
a first primer pair is used for the preamplification and at least one second primer pair is used for the at least one downstream detection reaction,
the at least one target DNA and the at least one reference DNA each have complementary sequence segments in relation to the primer sequences, and
the complementary sequence segments in relation to the at least one second primer pair lie within the segment between the complementary sequence segments in relation to the first primer pair.

12. The method as claimed in claim 10, further comprising:
using the nested PCR process for a point mutation detection; and
using a mutation-sensitive primer and/or a mutation-sensitive fluorescent probe for the at least one downstream detection reaction.

13. The method as claimed in claim 10, wherein:
the nested PCR process is a multiplex process for detecting at least two particular gene segments in a genome,
what is performed for a quantification of the preamplification is a control reaction in which a control exon from the genome is amplified,
the at least one target DNA and the at least one reference DNA are configured to match with the control exon, and
what are deduced from the quantification of the amplification of the control exon are the amounts in a case of the amplification of the gene segments to be detected during the preamplification.

14. A kit for performing a quantitative real-time polymerase chain reaction (PCR) for quantifying at least one deoxyribonucleic acid (DNA) segment, the kit comprising:
at least one target DNA which corresponds at least in parts to the at least one DNA segment to be quantified;
at least one reference DNA having a defined sequence and in a defined amount;
at least two different fluorescent probes of different sequence that generate signals at different wavelengths; and
optionally primers, deoxynucleotides, DNA polymerase, and/or buffer components, and
wherein the at least one target DNA and the at least one reference DNA have the same primer binding sites and different probe binding sites,
wherein at least one of the fluorescent probes is intended for binding with a segment of the at least one target DNA outside the primer binding sites, and
wherein at least one of the fluorescent probes is intended for binding with a segment of the at least one reference DNA outside the primer binding sites.

15. The kit as claimed in claim 14, wherein a GC content of the at least one target DNA and a GC content of the at least one reference DNA are identical with a deviation of up to 15%.
